(19) United States
(12) Patent Application Publication
(10) Pub. No.: US 2025/0359807 A1
(43) Pub. Date: Nov. 27, 2025

(54) METHOD AND SYSTEM FOR EVALUATING COGNITIVE FUNCTION BASED ON BRAIN WAVES

(71) Applicant: SEOUL NATIONAL UNIVERSITY R&DB FOUNDATION, Seoul (KR)

(72) Inventors: Chun Kee CHUNG, Seoul (KR); Jii KWON, Seoul (KR); Ye Jin PARK, Seoul (KR); Gyuwon LEE, Seoul (KR)

(21) Appl. No.: 19/211,449

(22) Filed: May 19, 2025

(30) Foreign Application Priority Data
May 24, 2024 (KR) 10-2024-0067565

(51) Int. Cl. A61B 5/38; A61B 5/00; G06F 40/35

(52) U.S. Cl. CPC A61B 5/38; A61B 5/4064; G06F 40/35

(57) ABSTRACT

One embodiment proposes a brainwave-based cognitive function evaluation method. The brainwave-based cognitive function evaluation method includes deriving a predicted response intended by an evaluation subject based on evaluation data including brainwaves of the evaluation subject for a specific task, generating a response accuracy including voice clarity, whether an answer matches, and answer meaning accuracy based on the predicted response and a preset correct answer for the specific task, and evaluating a cognitive function of the evaluation subject based on the response accuracy.

### CROSS-REFERENCE TO RELATED APPLICATION

This application is based on and claims priority under 35 U.S.C. § 119 to Korean Patent Application No. 10-2024-0067565, filed on May 24, 2024, in the Korean Intellectual Property Office, the disclosure of which is incorporated by reference herein in its entirety.

### BACKGROUND

### 1. Field

The present invention relates to a brainwave-based cognitive function evaluation system and method, and more specifically, to a method and system capable of quantitatively evaluating a cognitive function by predicting language intention from brain signals of a patient who has difficulty in verbal response and evaluating which function is impaired.

### 2. Description of the Related Art

It is necessary to evaluate language and cognitive functions of a patient who is conscious but may not speak or a patient who can speak but has poor memory and attention.

In order to determine a patient's memory, attention, and language abilities, minor mental state examination (MMSE) and Montreal cognitive assessment (MoCA) are performed conventionally. In this case, evaluation is made by asking a patient specific questions and determining whether the patient responds appropriately.

However, there is an issue that it is difficult to make a clear evaluation of memory and attention when a patient has difficulty in response due to a language disorder.

Also, because cognitive function evaluation is performed based on a patient's pure speech, there is a limitation in that it is difficult to clearly distinguish whether a patient's state of consciousness is normal or whether language ability is impaired due to a speech function problem.

Recently, prior art 1 for evaluating a cognitive function based on a brainwave has been presented. Prior art 1 discloses a technology for quantitatively evaluating a cognitive state from a response speed or response size of EEG after sensation is provided. However, prior art 1 has a limitation in that it is difficult to identify a cognitive function for high-level cognitive functions due to the evaluation of cognitive function in a passive state.

Also, prior art 2 for examining high-level cognitive functions based on brainwaves presents. Prior art 2 discloses a technology for extracting concentration, workload, and left/right brain balance from a change in brainwave after stimulation. However, prior art 2 evaluates how fast processing is performed for a given stimulus and has a limitation in that it is difficult to identify the cause of decreased state and ability to produce correct answers.

Accordingly, a method is required to determine whether the inability to speech is due to a decline in speech function caused by a decrease in physical activity or due to a disorder of consciousness.

### PRIOR ART DOCUMENTS

Prior art 1: Korean Patent Publication No. 10-2018-0021017 “EEG based cognitive function assessment device” (published on Feb. 28, 2018)
Prior art 2: Korean Patent No. 10-0751257 “Brain waves” (registered on Aug. 16, 2007)

### SUMMARY

The present invention provides a method and system capable of quantitatively evaluating a cognitive function by predicting language intention from brain signals of a patient who has difficulty in verbal response and evaluating which function is impaired.

Technical problems to be solved by the present invention are not limited to the technical problems described above, and other technical problems of the present invention can be derived from following descriptions.

An embodiment of the present disclosure provides a brainwave-based cognitive function evaluation method performed by a brainwave-based cognitive function evaluation system The brainwave-based cognitive function evaluation method includes deriving a predicted response intended by an evaluation subject based on evaluation data including brainwaves of the evaluation subject for a specific task, generating a response accuracy including voice clarity, whether an answer matches, and answer meaning accuracy based on the predicted response and a preset correct answer for the specific task, and evaluating a cognitive function of the evaluation subject based on the response accuracy.

Also, another embodiment of the present disclosure provides a brainwave-based cognitive function evaluation system. The brainwave-based cognitive function evaluation system includes at least one processor, and a memory electrically connected to the processor and storing at least one code executed by the processor. The memory stores a code that, when being executed by the processor, causes the processor to derive a predicted response intended by an evaluation subject based on evaluation data including brainwaves of the evaluation subject for a specific task, generate a response accuracy including voice clarity, whether an answer matches, and answer meaning accuracy based on the predicted response and a preset correct answer for the specific task, and evaluate a cognitive function of the evaluation subject based on the response accuracy.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating an example of a brainwave-based cognitive function evaluation system according to an embodiment of the present invention;

FIG. 2 is a diagram illustrating a brainwave-based cognitive function evaluation device according to an embodiment of the present invention;

FIG. 3 is a view illustrating a process of extracting voice clarity when a predicted response is in a spectrum form;

FIG. 4 is a diagram illustrating a process of determining answer meaning accuracy from brainwaves;

FIG. 5 is a flowchart illustrating a sequence of a brainwave-based cognitive function evaluation method according to another embodiment of the present invention; and

FIG. 6 is a flowchart illustrating a cognitive function evaluation process.

### DETAILED DESCRIPTION

Hereafter, the present disclosure will be described in detail with reference to the accompanying drawings. However, the present disclosure may be implemented in many different forms and is not limited to the embodiments described herein. Also, the accompanying drawings are only for easy understanding of the embodiments disclosed in the present specification, and the technical ideas disclosed in the present specification are not limited by the accompanying drawings. All terms, which include technical and scientific terms used herein, should be interpreted as having the meaning generally understood by a person of ordinary skill in the art to which the present disclosure belongs. Terms defined in advance should be interpreted as having additional meanings consistent with the relevant technical literature and the present disclosure, and should not be interpreted in a very ideal or restrictive sense unless otherwise defined.

In order to clearly describe the present disclosure in the drawings, parts irrelevant to the descriptions are omitted, and a size, a shape, and a form of each component illustrated in the drawings may be variously modified. The same or similar reference numerals are assigned to the same or similar portions throughout the specification.

Suffixes “shape”, “layer”, “function”, and so on for the components used in the following description are given or used interchangeably in consideration of case of writing the specification, and do not have meanings or roles that are distinguished from each other by themselves. Also, in describing the embodiments disclosed in the present specification, when it is determined that a detailed descriptions of related known technologies may obscure the gist of the embodiments disclosed in the present specification, the detailed descriptions are omitted.

Throughout the specification, when a portion is said to be “connected (coupled, in contact with, or combined)” with another portion, this includes not only a case where it is “directly connected (coupled, in contact with, or combined)””, but also a case where there is another member therebetween. Also, when a portion “includes (comprises or provides)” a certain component, this does not exclude other components, and means to “include (comprise or provide)” other components unless otherwise described.

Terms indicating ordinal numbers, such as first and second, used in the present specification are used only for the purpose of distinguishing one component from another component and do not limit the order or relationship of the components. For example, the first component of the present disclosure may be referred to as the second component, and similarly, the second element may also be referred to as the first component. As used herein, singular forms should be construed to include plural forms as well, unless the opposite is clearly indicated.

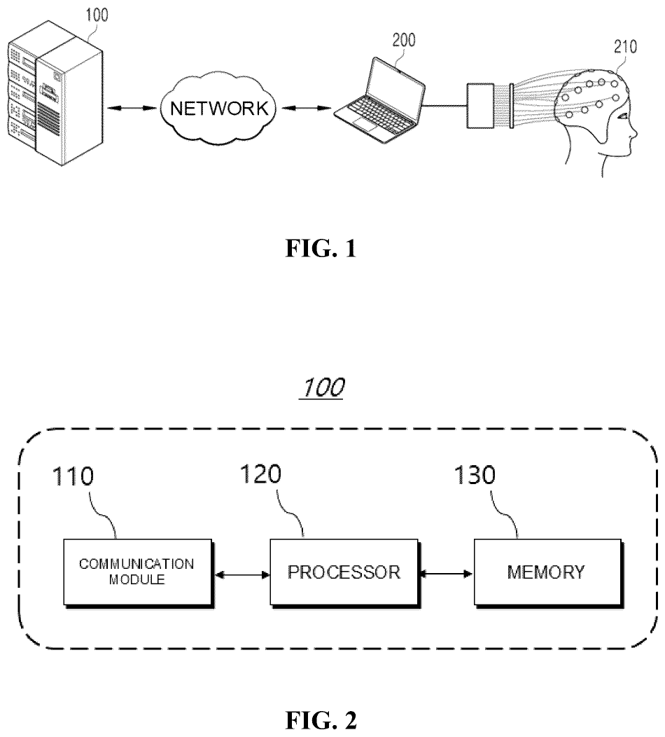

FIG. 1 is a diagram illustrating an example of a brainwave-based cognitive function evaluation system according to an embodiment of the present invention. Hereinafter, an environment for performing a brainwave-based cognitive function evaluation device (hereinafter referred to as a “cognitive function evaluation device”) or method according to an embodiment of the present invention is described below with reference to FIG. 1.

The brainwave-based cognitive function evaluation system according to the present embodiment may include a cognitive function evaluation device 100 and a brainwave measurement device 200.

The brainwave measurement device 200 may measure a brainwave including electroencephalography (EEG) from a target object and may receive a brainwave from an electrode cap (210) in which a plurality of electrodes are regularly arranged at regular intervals in a flexible hat according to the international 10-20 system or from a plurality of electrodes arranged on the head of a target object according to the international 10-20 system. The International 10-20 system is an international standard on a placement of electrodes that are attached or bonded to the scalp to determine a system of lines for placement of electrodes by using a distance between bone landmarks on the head. Electrodes for measuring brainwaves can be arranged at intervals of 10% or 20% of the total length of the lines.

In the present specification, a brainwave is described by using EEG as an example, but the brainwave includes all electrical and magnetic signals generated from brain neurons, such as magnetoencephalography (MEG) and electrocorticogram (ECoG), or images obtained by capturing the brain.

The brainwave measurement device 200 may be implemented by being included in the cognitive function evaluation device 100 or implemented separately from the cognitive function evaluation device 100. When the brainwave measurement device 200 is implemented separately from the cognitive function evaluation device 100, the cognitive function evaluation device 100 can receive brainwaves measured from a plurality of electrodes mounted on a target object from the brainwave measurement device 200 through a network or a wired/wireless interface.

Also, the brainwaves measured by the brainwave measurement device 200 may include a form of an image. For example, the brainwave may be an electroencephalogram topography generated based on an electroencephalogram. In the present specification, brainwaves may include not only electrical and magnetic signals generated from the brain's nerve cells, such as EEG, ECOG, or MEG, but also a topography generated based on the electrical and magnetic signals, and can be measured in various ways regardless of invasiveness or non-invasiveness. Also, a brainwave can be a brain image obtained by capturing the brain for a certain period of time, such as fMRI. The cognitive function evaluation device 100 may receive brainwaves of an evaluation

subject which are acquired by the brainwave measurement device 200, derive a predicted response based on evaluation data including the brainwaves, generate response accuracy including voice clarity, whether an answer matches, and answer meaning accuracy based on the predicted response and a preset correct answer, and evaluate a cognitive function of the evaluation subject based on the response accuracy.

In this case, the predicted response can be in the form of voice of the evaluation target object, and can be in the form of text or spectrum.

In order to derive a predicted response, a position and size of a formant expressed from the movement of a vocal cord can be predicted and analyzed in a motor cortex area of the brain. In this case, a long short-term memory (LSTM), a transformer, or a deep learning model can be used for time series analysis, or a support vector machine (SVM) model or a latent Dirichlet allocation (LDA) model can be used to quantize a formant into a specific bin and then predict probability of entering the bin.

Also, the number of consonants, vowels, and syllables to be uttered can be predicted and analyzed in a temporal lobe area of the brain. In this case, an LSTM, a transformer, a SVM model, or a k-nearest neighbor (KNN) model can be used to classify consonants, vowels, and syllables.

Also, a position of the formant predicted from the brainwaves can be converted into two dimensions to determine the probability of each vowel or the probability of the consonant or vowel.

When deriving a predicted response, semantic intent of the response can also be predicted. In this case, a prediction form of the semantic intent can be a category, an embedding vector, or a combination of the category and the embedding vector.

When predicting the semantic intent, a specific category and an embedding vector can be predicted in parallel from brainwaves, and then vectors for each word can be obtained again from a word embedding model for specific words within a category, and semantic prediction can be performed through a comparison of spatial similarity with the embedding vector predicted from brainwaves.

FIG. 2 is a diagram illustrating the brainwave-based cognitive function evaluation device 100 according to an embodiment of the present invention. Hereinafter, a configuration of the cognitive function evaluation device 100 is described with reference to FIG. 2.

The cognitive function evaluation device 100 may include a communication module 110 for interfacing or communicating with the brainwave measurement device 200 or electrodes for measuring brain signals, and the communication module 110 may include a mobile communication module, a wireless Internet module, or a short-range communication module as a network interface. Also, the cognitive function evaluation device 100 may be connected to the brainwave measurement device 200 through a communication network.

The communication module 110 may include a device including hardware and software required to transmit and receive signals, such as control signals or data signals through wired or wireless connections with other network devices.

The communication module 110 transmits and receives wireless signals to and from at least one of a base station, an external terminal, and a server on a mobile communication network constructed according to technical standards or communication methods (for example, global system for mobile communication (GSM), code division multi access (CDMA), CDMA2000, enhanced voice-data optimized or enhanced voice-data only (EV-DO), wideband CDMA (WCDMA), high speed downlink packet access (HSDPA), high speed uplink packet access (HSUPA), long term evolution (LTE), long term evolution-advanced (LTEA), and so on) for mobile communication used in a mobile communication module.

The wireless Internet module refers to a module for wireless Internet access and can be built into or externally installed in the cognitive function evaluation device 100. The wireless Internet module is configured to transmit and receive wireless signals in a communication network according to wireless Internet technologies.

The wireless Internet technologies include, for example, wireless LAN (WLAN), wireless-fidelity (Wi-Fi), Wi-Fi direct, digital living network alliance (DLNA), wireless broadband (WiBro), world Interoperability for microwave access (WiMAX), high speed downlink packet access (HSDPA), high speed uplink packet access (HSUPA), long term evolution (LTE), long term evolution-advanced (LTE-A), and so on.

The short-range communication module is for a short-range communication and can support the short-range communication by using at least one of technologies, such as Bluetooth™, radio frequency identification (RFID), infrared data association (IrDA), ultra wideband (UWB), ZigBee, near field communication (NFC), Wi-Fi, Wi-Fi Direct, and wireless universal serial bus (USB).

The cognitive function evaluation device 100 may include an interface unit for providing a user input or notification to a user, and the interface unit may include an optical output module, such as a display or light emitting diode (LED), or a voice output module, such as a speaker. The interface unit may include a mechanical input unit (or, a mechanical key, a dome switch, a jog wheel, a jog switch, or so on) and a touch input unit. For example, the touch input unit may include a virtual key, a soft key, or a visual key displayed on a touch screen through software processing, or a touch key placed on a component other than the touch screen.

The cognitive function evaluation device 100 may be implemented in the form of a server, a computing device, or various smart devices, and may operate in a cloud computing service model, such as software as a service (Saas), platform as a service (PaaS), or infrastructure as a service (IaaS). Also, the cognitive function evaluation device 100 may be constructed in the form of a private cloud, a public cloud, or a hybrid cloud system, but the scope of the present invention is not limited thereto.

The cognitive function evaluation device 100 may further include a processor 120 and a memory 130.

The processor 120 may include various types of devices that control and process data. The processor 120 may indicate a data processing device which is built in hardware and includes a physically structured circuit to perform a function expressed by codes or commands included in a program.

For example, the processor 120 may be implemented in the form of a microprocessor, a central processing unit (CPU), a processor core, a multiprocessor, an application-specific integrated circuit (ASIC), a field programmable gate array (FPGA), or so on, but the scope of the present invention is not limited thereto.

The cognitive function evaluation device 100 may use the processor 120 to apply a trained learning model to a specific input and infer a result value. Also, the processor 120 may be used to train a machine learning-based learning model. The processor 120 may determine optimized model parameters of an artificial neural network by repeatedly training the artificial neural network by using various learning techniques, or may infer a result value by applying a learning model to an input according to the model parameters of the trained artificial neural network.

The machine learning-based learning model may include a neural network, such as a convolutional neural network (CNN) or region based CNN (R-CNN), a convolutional recursive neural network (C-RNN), a fast R-CNN, a faster R-CNN, region based fully convolutional network (R-FCN), a you only look once (YOLO) or single shot multi-box detector (SSD) structure, or may include a classifier based on a support vector network (SVN). Also, the learning model may be implemented with hardware, software, or a combination of hardware and software, and when a part or all of the learning model is implemented with software, one or more commands constituting the learning model may be stored in the memory 130.

The processor 120 performs an operation according to the code stored in the memory 130.

The memory 130 can store at least one of information and data input to the communication module 110, information and data required for functions performed by the processor 120, and data generated according to the execution of the processor 120.

The memory 130 should be interpreted as a general term for a nonvolatile storage device that maintains the stored information even when power is not supplied and a volatile storage device that requires power to maintain the stored information. The memory 130 may include a cloud storage, a solid state drive (SSD), magnetic storage media, or flash storage media in addition to a volatile storage device that requires power to maintain the stored information, but the scope of the present invention is not limited thereto.

The memory 130 is electrically connected to the processor 120 and stores at least one code that is executed by the processor 120. The memory 130 stores code that causes the processor 120 to perform the following functions and procedures when executed by the processor 120.

When the memory 130 is executed through the processor 120, the memory 130 stores code that causes the processor 120 to derive a predicted response intended by an evaluation subject based on evaluation data including brainwaves of the evaluation subject on a specific task, generate response accuracy including voice clarity, whether an answer matches, and answer meaning accuracy based on the predicted response and a preset correct answer for the specific task, and perform evaluation on a cognitive function of the evaluation subject based on the response accuracy. In this case, the evaluation data further includes a vocal response of the evaluation subject for a specific task, and the response accuracy further includes a vocal response accuracy.

Also, when the predicted response is in the form of a spectrum, the memory 130 may further store code that causes the processor 120 to implement a waveform from the spectrum, obtain the number of peaks from the waveforms, and extract voice clarity based on whether the number of peaks is equal to the number of syllables of the preset correct answer.

Also, the memory 130 may further store code that causes the processor 120 to provide an individual evaluation result in which voice clarity is distinguished from answer meaning accuracy, or a comprehensive evaluation result in which voice clarity is combined with answer meaning accuracy.

Also, the memory 130 may further store code that causes the processor 120 to determine that a speech function is abnormal when the voice clarity is lower than a preset first value, determine that the speech function is normal when the voice clarity exceeds the preset first value, determine that a language function is abnormal when the answer meaning accuracy is lower than a preset second value, and determine that the language function is normal when the answer meaning accuracy exceeds the preset second value.

The cognitive function evaluation device 100 may further include a brain signal processing unit that performs pre-processing of a brain signal, and the brain signal processing unit may be implemented as a part of the processor 120 or implemented separately from the processor 120. Also, the brain signal processing unit may be implemented with a digital signal processor (DSP) chip or a graphics processing unit (GPU).

In this case, when a brain signal is an electroencephalogram, the brain signal processing unit may perform amplification and filtering, and remove noise or artifacts. Alternatively, the brain signal processing unit may perform an algorithm, such as Fourier analysis or wavelet analysis. Also, when a brain signal is a brain image, preprocessing of the brain image may be performed through an image processing algorithm.

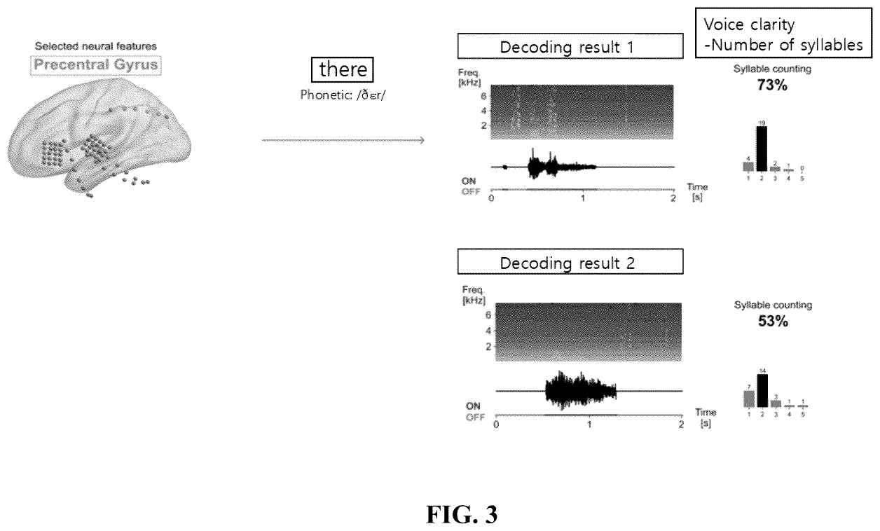

FIG. 3 is a view illustrating a process of extracting voice clarity when the predicted response is in a spectrum form.

Referring to FIG. 3, a process of extracting voice clarity when the predicted response is in the form of spectrum may include a process of implementing a waveform from the spectrum, obtaining the number of peaks from the waveform, and determining whether the number of peaks is equal to the number of syllables of a preset correct answer.

Also, the process of extracting the voice clarity may further include a process of extracting an envelope of a decoded waveform and determining whether the number of peaks of the envelope is equal to the number of syllables of correct answer.

Also, the predicted response in a spectrum form and the waveform implemented from the spectrum may include a value obtained by predicting a word for speech or a lip-shaped speech without sound based on a high gamma signal of a brainwave.

Also, the process of extracting voice clarity may include a process of measuring a ratio between a voice signal and a noise signal by using a speech transmission index (STI) or a speech intelligibility index (SII).

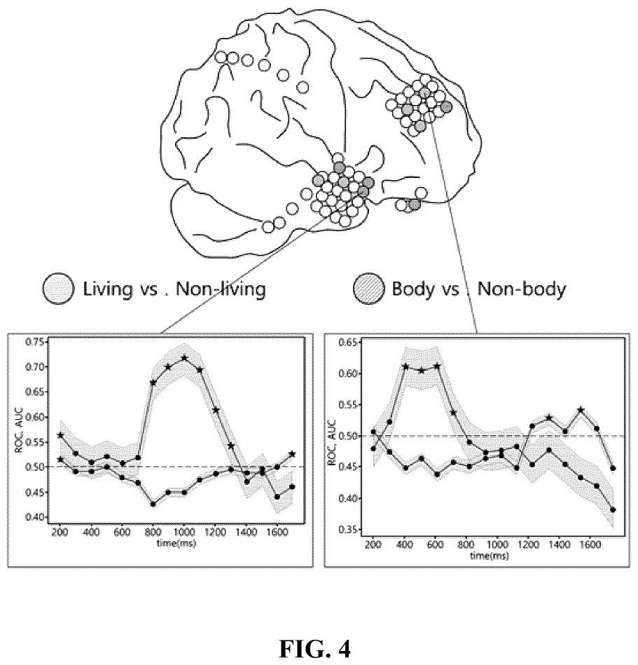

FIG. 4 is a diagram illustrating a process of determining answer meaning accuracy from brainwaves.

Referring to FIG. 4, it is possible to understand the meaning of an answer of an evaluation subject through brainwaves before hearing a question and answering the question. More specifically, when the evaluation subject answers with a word with a different meaning from the correct answer, it is possible to predict the meaning based on brainwaves by checking that brainwaves corresponding to an opposite meaning category are formed.

Also, even without using EEG, the meaning of a predicted word can be extracted based on a word embedding model, such as Word2Vec or GloVe, or a large language model (LLM), such as GPT, and the answer meaning accuracy can be generated based thereon.

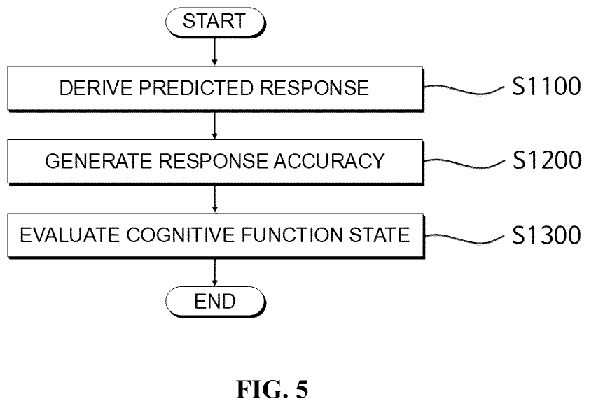

FIG. 5 is a flowchart illustrating a sequence of a brainwave-based cognitive function evaluation method according to another embodiment of the present invention.

The cognitive function evaluation method described below can be performed by the cognitive function evaluation device 100 described above with reference to FIGS. 1 to 4. Therefore, the descriptions of the embodiments of the present invention described above with reference to FIGS. 1 to 4 can be equally applied to the embodiments described below, and redundant descriptions thereof are omitted below. The operations described below do not have to be performed sequentially, and the order of the operations can be set in various ways, and the operations can be performed almost simultaneously.

Referring to FIG. 5, the brainwave-based cognitive function evaluation method includes operation S1100 of deriving a predicted response, operation S1200 of generating a response accuracy, and operation S1300 of evaluating a cognitive function.

The operation S1100 of deriving the predicted response is an operation of presenting a specific task to an evaluation subject and deriving a predicted response intended by the evaluation subject based on evaluation data including brainwaves of the evaluation subject for the specific task. In this case, a patient's brainwave includes all electrical and magnetic signals generated from brain cells, such as EEG, ECOG, and MEG, or all images obtained by capturing the brain.

Also, the evaluation data further includes a vocal response of the evaluation subject for the specific task, and a response accuracy may further include a vocal response accuracy.

Also, the predicted response derived from the evaluation data may be in any one of the forms of voice, text, and spectrum, but the scope of the present invention is not limited thereto.

Operation S1200 of generating a response accuracy is an operation of generating response accuracy including voice clarity, whether an answer matches, and an answer meaning accuracy from the predicted response and a preset correct answer for a specific task. In this case, when the predicted response is in the form of spectrum, the voice clarity can be extracted based on whether the number of peaks is equal to the number of syllables of the preset correct answer by implementing a waveform from the spectrum and obtaining the number of peaks from the waveform.

In this case, the specific task can be any one of a question with one correct answer, a question with two or more correct answers, and a question with no correct answer. When the specific task is a question with no correct answer, the correct answer can be extracted from an artificial intelligence-based language model, such as a generative pre-trained transformer (GPT), Liama, BERT, or n-gram, but the scope of the present invention is not limited thereto.

The voice clarity is an indicator that evaluates how clearly the voice is intended to be pronounced, regardless of whether the task is correct or not. The voice clarity can be extracted based on the clarity of distinguishment between syllables for the predicted answer based on brainwaves, extracted based on intonation, stress, and naturalness of a pattern, extracted by determining how much the consistency and stability of a fundamental frequency and the arrangement of a formant match the standard pronunciation, or determined by considering all the factors in combination, but the scope of the present invention is not limited thereto.

Whether the answer matches is an indicator for evaluating whether a predicted answer of the evaluation subject matches the correct answer of a task.

The answer meaning accuracy is an indicator for evaluating how much the predicted response of the evaluation subject matches the meaning of the correct answer of a task. In this case, an answer of the evaluation subject and a meaning vector of the correct answer can be found using a natural language processing technology, such as, Word2Vec and GloVe, and meaning similarity can be extracted from a deep learning model, such as cosine similarity, Jacard similarity, Levenshtein distance, or BERT, but the scope of the present invention is not limited thereto.

Operation S1300 of evaluating a cognitive function is an operation of evaluating a cognitive function of an evaluation subject based on the generated response accuracy. The evaluation of a cognitive function may include individual evaluations in which voice clarity is distinguished from answer meaning accuracy, or a comprehensive evaluation result in which voice clarity is combined with answer meaning accuracy.

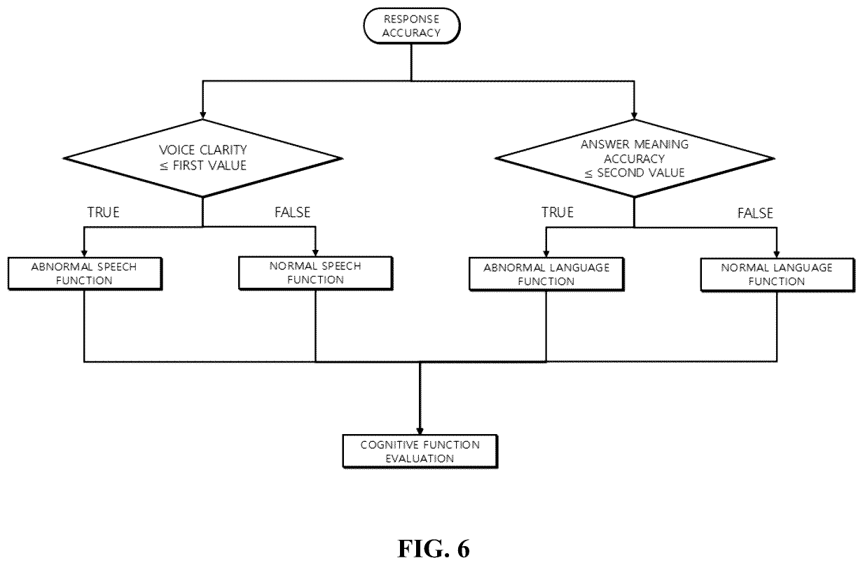

FIG. 6 is a flowchart illustrating a cognitive function evaluation process in detail.

Referring to FIG. 6, when the voice clarity is lower than a preset first value, a speech function is determined to be abnormal, and when the voice clarity exceeds the preset first value, the speech function is determined to be normal, and when the answer meaning accuracy is lower than a preset second value, a language function is determined to be abnormal, and when the answer meaning accuracy exceeds the preset second value, the language function is determined to be normal.

According to the solution to the problem of the present invention described above, a cognitive function can be quantitatively evaluated by using only brainwaves of a patient even when the patient may not act or speak.

Also, a language response to a specific task can be predicted, and accuracy can be extracted by dividing language into a phonetic dimension and a semantic dimension.

Also, it is possible to clearly distinguish what kind of disability a target object has.

Also, it is possible to predict a patient's speech intention even when there is no actual speech.

Also, language rehabilitation for patients with speech disorders and language disorders can be performed.

Effects of the present disclosure are not limited to the effects described above, and include all effects understood from the descriptions.

The cognitive function evaluation method of the embodiments of the present disclosure described above may be implemented in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

Those skilled in the art to which the present disclosure belongs will understand that the present disclosure may be easily modified into another specific form based on the descriptions given above without changing the technical idea or essential features of the present disclosure. Therefore, the embodiments described above should be understood as illustrative in all respects and not limiting. The scope of the present disclosure is indicated by the claims described below, and all changes or modified forms derived from the meaning, scope of the claims, and their equivalent concepts should be interpreted as being included in the scope of the present disclosure. The scope of the present application is indicated by the claims described below rather than the detailed description above, and all changes or modified forms derived from the meaning, scope of the claims, and their equivalent concepts should be interpreted as being included in the scope of the present application.

What is claimed is:

1. A brainwave-based cognitive function evaluation method performed by a brainwave-based cognitive function evaluation system, the brainwave-based cognitive function evaluation method comprising:
deriving a predicted response intended by an evaluation subject based on evaluation data including brainwaves of the evaluation subject for a specific task;
generating a response accuracy including voice clarity, whether an answer matches, and answer meaning accuracy based on the predicted response and a preset correct answer for the specific task; and
evaluating a cognitive function of the evaluation subject based on the response accuracy.

2. The brainwave-based cognitive function evaluation method of claim 1, wherein
the evaluation data includes a vocal response of the evaluation subject for the specific task, and
the response accuracy includes a voice response accuracy.

3. The brainwave-based cognitive function evaluation method of claim 1, wherein
the predicted response has any one form of voice, text, and spectrum.

4. The brainwave-based cognitive function evaluation method of claim 3, wherein,
when the predicted response is in a form of spectrum, the voice clarity is extracted based on whether a number of peaks is equal to a number of syllables of the preset correct answer by implementing a waveform from the spectrum and obtaining the number of peaks from the waveform.

5. The brainwave-based cognitive function evaluation method of claim 1, wherein
the specific task is any one of a question with one correct answer, a question with two or more correct answers, and a question with no correct answer, and
when the specific task is a question with no correct answer, a correct answer to the question is generated from an artificial intelligence based large language model (LLM).

6. The brainwave-based cognitive function evaluation method of claim 1, wherein
evaluation for the cognitive function includes a result of individual evaluation in which the voice clarity is distinguished from the answer meaning accuracy, or a result of comprehensive evaluation in which the voice clarity is combined with the answer meaning accuracy.

7. The brainwave-based cognitive function evaluation method of claim 1, wherein, in the evaluating of the cognitive function,
when the voice clarity is lower than or equal to a preset first value, a speech function is determined to be abnormal, and when the voice clarity exceeds the preset first value, the speech function is determined to be normal, and
when the answer meaning accuracy is lower than or equal to a preset second value, a language function is determined to be abnormal, and when the answer meaning accuracy exceeds the preset second value, the language function is determined to be normal.

8. A brainwave-based cognitive function evaluation system comprising:
at least one processor; and
a memory electrically connected to the processor and storing at least one code executed by the processor,
wherein the memory stores a code that, when being executed by the processor, causes the processor to derive a predicted response intended by an evaluation subject based on evaluation data including brainwaves of the evaluation subject for a specific task, generate a response accuracy including voice clarity, whether an answer matches, and answer meaning accuracy based on the predicted response and a preset correct answer for the specific task, and evaluate a cognitive function of the evaluation subject based on the response accuracy.

9. The brainwave-based cognitive function evaluation system of claim 8, wherein
the evaluation data includes a vocal response of the evaluation subject for the specific task, and
the response accuracy includes a voice response accuracy.

10. The brainwave-based cognitive function evaluation system of claim 8, wherein
the memory further stores a code that, when the predicted response is in a form of spectrum, causes the processor to extract the voice clarity based on whether a number of peaks is equal to a number of syllables of the preset correct answer by implementing a waveform from the spectrum and obtaining the number of peaks from the waveform.

11. The brainwave-based cognitive function evaluation system of claim 8, wherein
the memory further stores a code that causes the processor to provide a result of individual evaluation in which the voice clarity is distinguished from the answer meaning accuracy, or a result of comprehensive evaluation in which the voice clarity is combined with the answer meaning accuracy.

12. The brainwave-based cognitive function evaluation system of claim 8, wherein
the memory further stores a code that causes the processor to determine a speech function to be abnormal when the voice clarity is lower than or equal to a preset first value, determine the speech function to be normal when the voice clarity exceeds the preset first value, determine a language function to be abnormal when the answer meaning accuracy is lower than or equal to a preset second value, and determine the language function to be normal when the answer meaning accuracy exceeds the preset second value.
